(19) United States
(12) Patent Application Publication
(10) Pub. No.: US 2005/0100570 A1
(43) Pub. Date: May 12, 2005

(54) Multi-phase personal care composition

(75) Inventors: Karl Shiqing Wei, Mason, OH (US); Cheyne Pohlman Thomas, Independence, KY (US); Rebecca Ann Taylor, Cincinnati, OH (US); Paul Robert Tanner, Lebanon, OH (US); Qing Stella, Cincinnati, OH (US); Edward Dewey Smith III, Mason, OH (US); Mannie Lee Clapp, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/963,166

(22) Filed: Oct. 12, 2004

Related U.S. Application Data
(63) Continuation-in-part of application No. 10/841,174, filed on May 7, 2004.
(60) Provisional application No. 60/542,710, filed on Feb. 6, 2004.
(60) Provisional application No. 60/469,075, filed on May 8, 2003.
(60) Provisional application No. 60/469,566, filed on May 9, 2003.
(60) Provisional application No. 60/515,173, filed on Oct. 28, 2003.
(60) Provisional application No. 60/532,798, filed on Dec. 24, 2003.

(51) Int. Cl. A61K 7/021; A61K 7/00

(52) U.S. Cl. 424/401; 424/63

(57) ABSTRACT

The present invention relates to multi-phase personal care composition containing at least two visually distinct phases wherein at least one phase contains a particle. The phases form a pattern and are packaged in physical contact while remaining stable over time.

### CROSS REFERENCE TO RELATED APPLICATION(S)

This application is a Continuation-In-Part of Application No. 10/841,174, filed May 7, 2004, which claims the benefit of U.S. Provisional Application Nos. 60/542,710, filed Feb. 6, 2004; 60/469,075, filed May 8, 2003; 60/469,566, filed May 9, 2003; 60/515,173, filed Oct. 28, 2003; and 60/532,798, filed Dec. 24, 2003.

### FIELD OF THE INVENTION

The present invention relates to multi-phase personal care composition comprising at least two visually distinct phases wherein the two phases are packaged in physical contact while remaining stable over time.

### BACKGROUND OF THE INVENTION

Personal care compositions are well known and widely used. These compositions have long been employed to cleanse and moisturize skin, deliver actives, hide imperfections and to reduce the oiliness/shine associated with sebum. Personal care compositions have also been used to alter the color and appearance of skin.

While the compositions and disclosures of the prior art provide useful advances in the art of personal care compositions, additionally, there remains the need for improved personal care compositions that deliver immediate improvements in appearance and skin feel that will effectively deposit on all parts of the body. The compositions also need to be non-greasy and easy to apply. Therefore, it is desirable to provide a personal care composition comprising a select level and blend of particles to provide a unique level of light reflectance, color shift or exfoliation to increase the radiance across all skin types and improve skin cleansing. Furthermore, it is desirable to provide a personal care composition comprising particles to maximize sheen and lustre on the skin.

One attempt at providing a multiple phase composition from a personal care product while maintaining stability has been the use of dual-chamber packaging. These packages comprise primarily separate cleansing compositions and conditioning compositions contained in different compartments, and allow for the co-dispensing of the two in a single or dual stream. The separate conditioning and cleansing compositions thus remain physically separate and stable during prolonged storage and just prior to application, but then mix during or after dispensing to provide conditioning and cleansing benefits from a physically stable system. Although such dual-chamber delivery systems provide improved conditioning benefits over the use of conventional systems, it is often difficult to achieve consistent and uniform performance because of the uneven dispensing ratio between the cleansing phase and the conditioning phase from these dual-chamber packages. Additionally, these packaging systems add considerable cost to the finished product.

It is further desirable to deliver the above skin conditioning, cleansing and appearance benefits via an in-the-shower or in-the-bath composition. Accordingly, the need still remains for stable multi-phase personal care composition that provides cleansing with increased lather longevity and improved lathering characteristics, and skin benefits such as silky skin feel, improved soft skin feel, and improved smooth skin feel and provides a unique level of light reflectance, colour shift and exfoliation to increase the radiance across all skin types. It has now been found that multi-phase personal cleansing compositions comprising at least two phases in physical contact that remain stable over time can be formulated.

It is therefore an object of the present invention to provide a multi-phase personal care composition comprising at least two visually distinct phases wherein at least one phase comprises particles wherein the two phases are packaged in physical contact while remaining stable, wherein the compositions can be formulated to provide improved cosmetics and skin feel during and after application while also providing excellent skin conditioning and cleansing benefits and deliver radiance across all skin types. It has been found that such a composition can be formulated with sufficiently high levels of benefit agents without compromising product lather performance and stability.

### SUMMARY OF THE INVENTION

The present invention relates to a multi-phase personal care composition comprising; at least two visually distinct phases;
wherein the phases form a pattern;
wherein at least one phase comprises a particle;
wherein said particle is present at a cosmetically efficacious level; and
wherein said two phases are packaged in physical contact with one another and maintain stability.

The present invention further relates to a multi-phase personal care composition comprising at least two phases wherein at least one phase contains a colorant, wherein both phases are packed in a single package such that the two phases form a pattern visible to the naked eye.

The present invention further relates to a multi-phase personal care composition comprising; at least two visually distinct phases;
wherein the phases form a pattern;
wherein at least one phase comprises a particle;
wherein a ratio of a first phase to a second phase is about 90:10 to about 10:90; and
wherein said two phases are packaged in physical contact with one another and maintain stability.

The present invention further relates to a multi-phase personal care composition comprising:
a) a first phase comprising a cleansing phase comprising from about 1% to about 50%, by weight of the cleansing phase, of a surfactant selected from the group consisting of anionic surfactant, nonionic surfactant, zwitterionic surfactant, cationic surfactant, soap, and mixtures thereof;
wherein the cleansing phase is non-Newtonian shear thinning, has a viscosity of equal to or greater than about 3,000 cps, and/or has a Yield Point of at least about 0.1 Pa;
b) a benefit phase comprising a hydrophobic composition comprising from about 20% to about 100% by weight of the benefit phase of a hydrophobic material selected from the group consisting of lipids, hydrocarbons, fats, oils, hydrophobic plant extracts, fatty acids, essential oils, silicone oils, and mixtures thereof;
wherein said hydrophobic composition has a Vaughan Solubility Parameter of about 5 to about 15 and further wherein the weight ratio between the cleansing phase and the benefit phase is from about 1:9 to about 99:1 and the cleansing phase and benefit phase are in physical contact in the same package and remain stable in ambient conditions for at least about 180 days; and wherein the cleansing phase and benefit phase form a pattern of striped wherein the stripe size is at least about 0.1 mm in width and at least about 1 mm in length; wherein at least one phase comprises a particle, wherein said particle is preferably hydrophobically modified interference pigment.

The present invention is also directed to a method of cleansing, moisturizing and delivering skin benefit agents and particles to the skin by applying to the skin a composition as described above.

### DETAILED DESCRIPTION

The multi-phase personal care compositions of the present invention comprises at least two visually distinct phases; wherein the phases form a pattern; wherein at least one phase comprises a particle; wherein said particle is present at a cosmetically efficacious level; and wherein said two phases are packaged in physical contact with one another and maintain stability.

These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water.

The term “ambient conditions” as used herein, refers to surrounding conditions at one (1) atmosphere of pressure, 50% relative humidity, and 25° C.

The term “cosmetically efficacious level” as used herein, is a level conferring a benefit during use of the composition.

The term “Consistency value” or “k” as used herein is a measure of viscosity and is used in combination with Shear Index, to define viscosity for materials whose viscosity is a function of shear. The measurements are made at 25° C and the units are poise (equal to 100 cps).

The term “hydrophobically modified interference pigment” or “HMIP”, as used herein, means a portion of the interference pigment surface has been coated, including both physical and chemical bonding of molecules, with a hydrophobic material.

The term “interference pigment”, as used herein, means a pigment with pearl gloss prepared by coating the surface of a particle substrate material (generally platelet in shape) with a thin film. The thin film is a transparent or semitransparent material having a high refractive index. The higher refractive index material shows a pearl gloss resulting from mutual interfering action between reflection and incident light from the platelet substrate/coating layer interface and reflection of incident light from the surface of the coating layer.

By the term “multi-phased” or “multi-phase” as used herein, is meant that the at least two phases herein occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled and combinations thereof.

In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The size of the stripes is at least about 0.1 mm in width and 10 mm in length, preferably at least about 1 mm in width and at least 20 mm in length. The phases may be various different colors, or include particles, glitter or pearlescence.

The term “personal care composition” as used herein, refers to compositions intended for topical application to the skin or hair.

The term “phases” as used herein, refers to a domain or region of a composition having one average composition, as distinct from another region or domain having a different average composition, wherein the domains are visible to the naked eye. This would not preclude the distinct regions or domains from comprising two similar phases where one phase could comprise pigments, dyes, particles, and various optional ingredients, hence a region or domain of a different average composition.

The term “stable” as used herein, unless otherwise specified, refers to compositions that maintain at least two “separate” phases when sitting in physical contact at ambient conditions for a period of at least about 180 days. By “separate” is meant that there is substantially no mixing of the phases, observable to the naked eye, prior to dispensing of the composition.

The term “Shear Index” or “n” as used herein is a measure of viscosity and is used in combination with Consistency value, to define viscosity for materials whose viscosity is a function of shear. The measurements are made at 25° C. and the units are dimensionless.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The Vaughan Solubility Parameter (VSP) as used herein is a parameter used to define the solubility of hydrophobic compositions comprising hydrophobic materials. Vaughan Solubility parameters are well known in the various chemical and formulation arts and typically have a range of from about 5 to about 25 cal/cm³.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The multi-phase personal care compositions and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

### Product Form

The multi-phase personal care composition of the present invention is typically in the form of a liquid. The term “liquid” as used herein means that the composition is generally flowable to some degree. “Liquids”, therefore, can include liquid, semi-liquid, cream, lotion or gel compositions intended for topical application to skin. The compositions typically exhibit a viscosity of equal to or greater than about 3,000 cps to about 1,000,000 cps, as measured by the Viscosity Method described hereafter. These compositions contain at least two phases, which are described in greater detail hereinafter.

When evaluating the multi-phased personal care composition, by the methods described herein, preferably each individual phase is evaluated prior to combining, unless otherwise indicated in the individual methodology. However, if the phases are combined, each phase can be separated by centrifugation, ultracentrufigation, pipetting, filtering, washing and then the separate phase can be evaluated.

All of the product forms contemplated for purposes of defining the compositions and methods of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

### Phases

The multi-phase personal care compositions of the present invention comprising at least two phases, wherein in the composition can have a first phase a second phase and so on. The ratio of a first phase to a second phase is about 90:10 to about 10:90, preferably about 80:20 to about 20:80, more preferably about 70:30 to about 30:70, even more preferably about 60:40 to about 40:60, still more preferably about 50:50. Each phase could be one or more of the following nonlimiting examples including: a cleansing phase, a benefit phase, and a non-lathering structured aqueous phase,which are described in greater detail hereinafter.

1) Cleansing Phase

The multi-phase personal care compositions of the present invention can comprise a cleansing phase. The cleansing phase of the present invention comprises a surfactant suitable for application to the skin or hair. Suitable surfactants for use herein include any known or otherwise effective cleansing surfactant suitable for application to the skin, and which is otherwise compatible with the other essential ingredients in the cleansing phase of the compositions including water. These cleansing surfactants include anionic, nonionic, cationic, zwitterionic or amphoteric surfactants, or combinations thereof. The cleansing surfactant phase in the present invention exhibits Non-Newtonian shear thinning behavior. Preferably, the cleansing phase has a viscosity of greater than about 3,000 centipoise (“cps”), more preferably greater than about 5,000 cps, even more preferably greater than about 10,000 cps, and still more preferably greater than about 20,000 cps, as measured by the Viscosity Method described hereafter.

Preferably, the cleansing phase has a Yield Point of greater than about 0.1 Pascal (Pa), more preferably greater than about 0.5 Pascal, even more preferably greater than about 1.0 Pascal, still more preferably greater than about 2.0 Pascal, still even more preferably greater than about 5 Pascal, and even still even more preferably greater than about 10 Pascal as measured by the Yield Point Method described hereafter.

The cleansing phase of the multi-phase personal care composition preferably comprises a cleansing surfactant at concentrations ranging from about 1% to about 50%, more preferably from about 4% to about 30%, even more preferably from about 5% to about 25%, by weight of the cleansing phase. The preferred pH range of the cleansing phase is from about 5 to about 8, more preferably about 6.

The cleansing phase of the personal care compositions produces a Total Lather Volume of at least about 400 ml, preferably greater than about 500 ml, more preferably greater than about 600 ml, even more preferably greater than about 800 ml, still more preferably greater than about 1000 ml, and still even more preferably greater than about 1250 ml, as measured by the Lather Volume Test described in copending application Ser. No. 60/532,798 filed on Dec. 24, 2003. The lathering cleansing phase of the personal care compositions preferably produces a Flash Lather Volume of at least about 100 ml, preferably greater than about 200 ml, even more preferably greater than about 300 ml, as measured by the Lather Volume Test described in copending application Ser. No. 60/532,798 filed on Dec. 24, 2003.

Anionic surfactants suitable for use in the cleansing phase include alkyl and alkyl ether sulfates. These materials have the respective formula ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohols having from about 8 to about 24 carbon atoms. Preferably, R has from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with about 1 to about 10, preferably from about 3 to about 5, and more preferably with about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, preferably about 10 to about 18, carbon atoms; and M is a cation. Suitable examples are the salts of an organic sulfuric acid reaction product of a hydrocarbon of the methane series, including iso-, neo-, ineso-, and n-paraffins, having about.8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Other suitable surfactants are described in McCutcheon's, Emulsifiers and Detergents, 1989 Annual, published by M. C. Publishing Co., and in U.S. Pat. No. 3,929,678.

Preferred anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants may be used in some embodiments.

Additional surfactant from the classes of amphoteric, zwitterionic surfactant, cationic surfactant, and/or nonionic surfactant may be incorporated in the cleansing phase compositions.

Amphoteric surfactants suitable for use in the cleansing phase include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Zwitterionic surfactants suitable for use in the cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula: 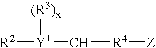
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Amphoacetates and diamphoacetates may also be used. 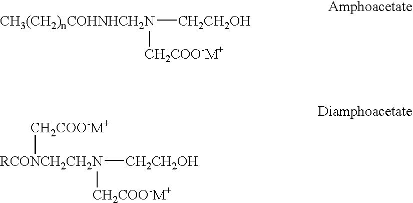
Amphoacetates and diamphoacetates conform to the formulas (above) where R is an aliphatic group of 8 to 18 carbon atoms. M is a cation such as sodium, potassium, ammonium, or substituted ammonium. Sodium lauroamphoacetate, sodium cocoamphoactetate, disodium lauroamphoacetate, and disodium cocodiamphoacetate are preferred in some embodiments.

Cationic surfactants can also be used in the cleansing phase, but are generally less preferred, and preferably represent less than about 5% by weight of the compositions.

Suitable nonionic surfactants for use in the aqueous cleansing phase include condensation products of alkylene oxide groups (hydrophilic in nature) with an organic hydrophobic compound, which may be aliphatic or alkyl aromatic in nature.

A) Structurant

The cleansing phase of the present compositions optionally, but preferably, further comprise about 0.1% to 10% by wt. of a structurant which functions in the compositions to form a thermodynamic phase, preferably a lamellar thermodynamic phase. It is believed the lamellar phase enhances the interfacial stability between the phases of the present compositions.

Suitable structurants include a fatty acid or ester derivatives thereof, a fatty alcohol, trihydroxystearin (available from Rheox, Inc. under the trade name THIXCIN® R), or polymethyacrylamidopropyl trimonium chloride (available from Rhodia under the trade name POLYCARE® 133). Preferably, the lamellar structurant is selected from lauric acid or trihydroxystearin.

In a preferred embodiment of the present invention, the surfactant for use in the cleansing phase exhibit Non-Newtonian shear thinning behavior (herein referred to as free flowing compositions) and can be mixtures of surfactants. Suitable surfactant mixtures can comprise water, at least one anionic surfactant, an electrolyte, and at least one alkanolamide. It has been found that by employing a cleansing phase exhibiting Non-Newtonian shear thinning behavior, the stability of the resulting multi-phased personal care composition can be increased. The alkanolamide if present has the general structure of: 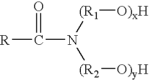
wherein R is C₈ to C₂₄, or preferably in some embodiments C₈ to C₂₂ or in other embodiments C₈ to C₁₈, saturated or unsaturated, straight chain or branched, aliphatic group; R₁ and R₂ are the same or different C₂-C₄ straight chain or branched aliphatic group; x is from 0 to 10; y is from 1 to 10; and wherein the sum of x and y is less than or equal to 10.

The amount of alkanolamide in the composition is typically about 0.1% to about 10%, by weight of the cleansing phase, and in some embodiments is preferably from about 2% to about 5%, by weight of the cleansing phase. Suitable alkanolamides include Cocamide MEA (Coco monethanolamide) and Cocamide MIPA (Coco monoisopropranolamide).

The electrolyte, if used, can be added per se to the composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride or sodium or ammonium sulfate. A preferred electrolyte is sodium chloride.

The electrolyte, when present, should be present in an amount, which facilitates formation of the free flowing composition. Generally, this amount is from about 0.1% by weight to about 15% by weight, preferably from about 1% to about 6% by weight of the cleansing phase, but may be varied if required.

In one embodiment of the present invention, the cleansing phase comprises an anionic surfactant (e.g. sodium trideceth sulfate), an amphoacetate surfactant (e.g. sodium lauroamphoacetate), and an alkanolamide (e.g. cocoamide MEA). The cleansing phase of this embodiment preferably further comprises an electrolyte (e.g. sodium chloride).

B) Isotropic

The cleansing phase of the present compositions may optionally comprise an isotropic thermodynamic phase. The isotropic thermodynamic phase comprises surfactant solutions which are composed of completely miscible components whose microstructure does not vary with distance or direction in the solution. Preferably, the surfactants found in the isotropic thermodynamic phase are the same as those mentioned previously in the lamellar thermodynamic phase. The isotropic thermodynamic phase of the multiphase personal care composition preferably has a viscosity in the range of about 3,000 to about 100,000 centipoises (cps) measured at 0.5 RPM using a Brookfield Cone and Plate viscometer with spindle number S41. More preferably the viscosity is about 10,000 to about 100,000 cps, even more preferably the viscosity is about 20,000 to about 80,000 cps.

C) Organic Cationic Deposition Polymer

The multi-phased personal care compositions of the present invention may additionally comprise an organic cationic deposition polymer in the cleansing phase as a deposition aid for the benefit agents described hereinafter. Concentrations of the cationic deposition polymer preferably range from about 0.025% to about 3%, more preferably from about 0.05% to about 2%, even more preferably from about 0.1% to about 1%, by weight of the cleansing phase composition.

Suitable cationic deposition polymers for use in the multi-phased personal care compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. The average molecular weight of the cationic deposition polymer is between about 5,000 to about 10 million, preferably at least about 100,000, more preferably at least about 200,000, but preferably not more than about 2 million, more preferably not more than about 1.5 million. The polymers also have a cationic charge density ranging from about 0.2 meq/gm to about 5 meq/gm, preferably at least about 0.4 meq/gm, more preferably at least about 0.6 meq/gm., at the pH of intended use of the personal cleansing composition, which pH will generally range from about pH 4 to about pH 9, preferably between about pH 5 and about pH 8.

The charge density can be controlled and adjusted in accordance with techniques well known in the art. As used herein the “charge density” of the cationic polymers is defined as the number of cationic sites per polymer gram atomic weight (molecular weight), and can be expressed in terms of meq/gram of cationic charge. In general, adjustment of the proportions of amine or quaternary ammonium moieties in the polymer, as well as pH of the multi-phased personal care compositions in the case of the amines, will affect the charge density.

Any anionic counterions can be use in association with the cationic deposition polymers so long as the polymers remain soluble in water, in the multi-phased personal care compositions, or in a coacervate phase of the multi-phased personal care compositions, and so long as the counterions are physically and chemically compatible with the essential components of the personal cleansing composition or do not otherwise unduly impair product performance, stability or aesthetics. Nonlimiting examples of such counterions include halides (e.g., chlorine, fluorine, bromine, iodine), sulfate and methlylsulfate.

Nonlimiting examples of cationic deposition polymers for use in the multi-phase personal care compositions include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M.

Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

The cationic polymers herein are either soluble in the cleansing phase, or preferably are soluble in a complex coacervate phase in the multi-phased personal care compositions formed by the cationic deposition polymer and the anionic surfactant component described hereinbefore. Complex coacervates of the cationic deposition polymer can also be formed with other charged materials in the multi-phased personal care compositions.

Coacervate formation is dependent upon a variety of criteria such as molecular weight, component concentration, and ratio of interacting ionic components, ionic strength (including, modification of ionic strength, for example, by addition of salts), charge density of the cationic and anionic components, pH, and temperature. Coacervate systems and the effect of these parameters have been described, for example, by J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid anti Interface Science, Vol. 140, No. 1, November 1990, pp 227-238, which descriptions are incorporated herein by reference.

It is believed to be particularly advantageous for the cationic deposition polymer to be present in the multi-phased personal care compositions in a coacervate phase, or to form a coacervate phase upon application or rinsing of the cleansing composition to or from the skin. Complex coacervates are believed to more readily deposit on the skin, which results in improved deposition of the benefit materials. Thus, in general, it is preferred that the cationic deposition polymer exists in the multi-phased personal care compositions as a coacervate phase or form a coacervate phase upon dilution. If not already a coacervate in the multi-phased personal care compositions, the cationic deposition polymer will preferably exist in a complex coacervate form in the multi-phased personal care compositions upon dilution with water.

Techniques for analysis of formation of complex coacervates are known in the art. For example, centrifugation analyses of the multi-phased personal care compositions, at any chosen stage of dilution, can be utilized to identify whether a coacervate phase has formed.

2) Benefit Phase

The multi-phase personal care compositions of the present invention can comprise a benefit phase. The benefit phase in the present invention is preferably anhydrous. The benefit phase comprises hydrophobic compositions comprising hydrophobic materials. The benefit phase comprises from about 20% to about 100%, preferably at least about 35%, most preferably at least about 50% of a hydrophobic material. The hydrophobic compositions suitable for use in the present invention have a Vaughan Solubility Parameter of from about 5 to about 15. The hydrophobic compositions are preferably selected among those having defined rheological properties as described hereinafter, including selected Consistency value (k) and Shear Index (n). These preferred rheological properties are especially useful in providing the multi-phased personal care compositions with improved deposition of hydrophobic materials on the skin.

A) Vaughan Solubility Parameter Value (VSP)

The hydrophobic compositions for use in the benefit phase of the multi-phase personal care composition has a Vaughan Solubility Parameter (VSP) of from about 5 to about 15, preferably from about 5 to about 10, more preferably from about 6 to about 9. These solubility parameters are well known in the formulation arts, and are defined by Vaughan in Cosmetics and Toiletries, Vol. 103, p47-69, October 1988.

Non-limiting examples of hydrophobic materials having VSP values ranging from about 5 to about 15 include the following:

VAUGHAN SOLUBILITY PARAMETERS*

| | |
|---|---|
| Cyclomethicone | 5.92 |
| Squalene | 6.03 |
| Petrolatum | 7.33 |
| Isopropyl Palmitate | 7.78 |
| Isopropyl Myristate | 8.02 |
| Castor Oil | 8.90 |
| Cholesterol | 9.55 |

As reported in Solubility, Effects in Product, Package, Penetration and Preservation, C. D. Vaughan, Cosmetics and Toiletries, Vol. 103, October 1988.

B) Rheology

The hydrophobic compositions for use in the benefit phase of the composition have a preferred rheology profile as defined by Consistency Value (k) and Shear Index (n). Preferred Consistency Value ranges are about 1-10,000 poise/(1/sec) (poise per inverse second), preferably about 10-5000 poise/(1/sec) and more preferably about 504000 poise/(1/sec). Shear Index ranges are about 0.025-0.9, preferably about 0.05-0.5 and more preferably about 0.09-0.4.

The hydrophobic composition can be characterized by Consistency Value (k) and Shear Index (n) values as defined by the above-described ranges, wherein these defined ranges are selected to provide enhanced deposition and reduced stickiness during and after application of the multi-phase personal care composition on hair or skin.

The Shear Index (n) and Consistency (k) Values are well known and accepted industry standards for reporting the viscosity (μ) profile of materials having a viscosity that varies with applied shear rate.

The viscosity (μ) for a hydrophobic composition can be characterized by either applying a shear rate and measuring the resultant shear stress or vice versa in a programmed manner using a rheometer, such as a TA Instruments AR2000 (TA Instruments, New Castle, Del., USA 19720). Viscosity is determined at different shear rates in the following manner. First, the hydrophobic composition is obtained which has the composition and properties as existing in the multi-phase personal care composition. That is, the composition is processed in a similar manner such that, for example, it is crystallized at approximately the same rate, if the sample contains crystals. An aliquot of the hydrophobic composition can be obtained prior to combining in the multiphase composition, as is common practice to those having skill in the art. Also, the hydrophobic composition can be recovered from the multi-phase personal care composition, for example by centrifuging, pipetting, sieving, rinsing, or other means to recover the hydrophobic composition. The AR2000 rheometer is programmed to shear the sample by ramping the stress from about 0.1 Pa to about 1,000 Pa over a 5 minute interval at 25 degrees Celsius. A 4 cm parallel plate geometry with a gap of 1 mm is common, although the gap can be increased or decreased as necessary, for example if the hydrophobic composition contains large particles, the gap may need to be larger. A shear rate of at least 100 1/seconds is obtained in the test, or the test is repeated with a higher final stress value while maintaining the programmed rate of stress increase at about 1.25 minutes per decade of stress. These results are fitted with the following well accepted power law model. Data in the sheared region are included, by plotting the viscosity and shear rate data on a log-log plot, and utilizing only the data in the region where shear rate is ascending and viscosity is descending in steady fashion. For example, an initial plateau region at low shear stress where little flow occurs is not considered. Typically, the viscosity between about 0.1-10.0 1/seconds shear rate is useful and enough data points are taken to fit to the well accepted power law model (see for instance: Chemical Engineering, by Coulson and Richardson, Pergamon, 1982 or Transport Phenomena, by Bird, Steward and Lightfoot, Wiley, 1960):
μ=k(γ′)⁽ⁿ⁻¹⁾

The value obtained for the log-log slope is (n-1) where n is the Shear Index and the value obtained for k is the Consistency Value in poise/(1/second). Petrolatum (Super White Protopet, Witco) for example, has a Consistency Value of about 2,000 poise/(1/second) and a Shear Index of about 0.2; and a blend of 50% petrolatum with 50% Hydrobrite 1000 mineral oil (Witco) has a Consistency Value of about 400 poise/(1/second) and a Shear Index of about 0.25.

The hydrophobic composition comprises hydrophobic materials. Nonlimiting examples of hydrophobic material suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, and combinations thereof.

Non-limiting examples of silicone oils suitable for use as hydrophobic materials herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use as hydrophobic materials herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as hydrophobic materials herein include acetylated monoglycerides.

Non-limiting examples of alkyl esters suitable for use as hydrophobic materials herein include isopropyl esters of fatty acids and long chain esters of long chain (i.e. C₁₀-C₂₄) fatty acids, e.g. cetyl ricinoleate, non-limiting examples of which incloude isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic materials herein include oleyl myristate, oleyl stearate, oleyl oleate, and combinations thereof.

Non-limiting examples of polyglycerin fatty acid esters suitable for use as hydrophobic materials herein include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic materials herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable hydrophobic materials include milk triglycerides (e.g., hydroxylated milk glyceride) and polyol fatty acid polyesters.

Still other suitable hydrophobic materials include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The benefit phase of the composition preferably can comprise one or more hydrophobic materials, wherein at least 20% by weight of the hydrophobic materials are selected from petrolatum, mineral oil, sunflower seed oil, micro-crystalline waxes, paraffins, ozokerite, polyethylene, polybutene, polydecene and perhydrosqualene dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, castor oil, soy bean oil, maleated soy bean oil, safflower oil, cotton seed oil, corn oil, walnut oil, peanut oil, olive oil, cod liver oil, almond oil, avocado oil, palm oil and sesame oil, and combinations thereof. More preferably, at least about 50% by weight of the hydrophobic materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic skin conditioning agent is preferably selected from: isopropyl palmitate, cetyl riconoleate, octyl isononanoate, octyl palmitate, isocetyl stearate, hydroxylated milk glyceride and combinations thereof.

3) Non-Lathering Structured Aqueous Phase

The multi-phase personal care compositions of the present invention can comprise a non-lathering structured aqueous phase. The non-lathering structured aqueous phase of the compositions of the present invention comprises a water structurant and water. The non-lathering structured aqueous phase can be hydrophilic and in a preferred embodiment the non-lathering structured aqueous phase is a hydrophilic gelled water phase. In addition, the non-lathering structured aqueous phase of the present invention typically comprises less than about 5%, preferably less than about 3%, and more preferably less than about 1%, by weight of the non-lathering structured aqueous phase, of a surfactant. In one embodiment of the present invention, the non-lathering structured aqueous phase is free of surfactant.

The non-lathering structured aqueous phase of the personal care compositions preferably produces a Total Lather Volume of no greater than about 350 ml, more preferably no greater than about 330 ml, even more preferably no greater than about 300 ml, as measured by the Lather Volume Test described in copending application Ser. No. 60/532,798 filed on Dec. 24, 2003. The non-lathering structured aqueous phase of the personal care compositions preferably produces a Flash Lather Volume of no greater than about 150 ml, preferably no greater than about 130 ml, even more preferably no greater than about 100 ml, as measured by the Lather Volume Test described in copending application Ser. No. 60/532,798 filed on Dec. 24, 2003.

Preferably, the non-lathering structured aqueous phase exhibits a Yield Point of at least about 0.1 Pa, preferably at least about 1 Pa, more preferably at least about 10 Pa, as measured by the Yield Point Method described hereafter. Preferably, the non-lathering structured aqueous phase exhibits a Water Mobility of less than about 2.5 seconds, more preferably less than about 2 seconds, and even more preferably less than about 1 second, as measured by the Water Mobility Method described in copending applications Ser. No. 60/532,798 filed on Dec. 24, 2003.

Preferably, the non-lathering structured aqueous phase exhibits a Correlated Haze of less than about 50% Correlated Haze, more preferably less than about 30% Correlated Haze, even more preferably less than about 20% Correlated Haze, and still more preferably less than about 10% Correlated Haze as measured by the Correlated Haze Index Method described hereafter.

The non-lathering structured aqueous phase has a preferred rheology profile as defined by Consistency Value (k) and Shear Index (n). Preferred Consistency Values of the non-lathering structured aqueous phase are from about 10 to about 100,000 poise/(1/s), preferably from about 10 to about 10,000 poise/(1/s), and more preferably from about 100 to about 1,000 poise/(1/s). The Shear Index of the non-lathering structured aqueous phase typically ranges from about 0.1 to about 0.8, preferably from about 0.1 to about 0.5, and more preferably from about 0.20 to about 0.4.

The Shear Index (n) and Consistency Value (k) are well-known and accepted industry standards for reporting the viscosity profile of compositions having a viscosity that is a function of an applied shear rate. The methodology used to obtain these values was described in greater detail previously.

The non-lathering structured aqueous phase of the present invention comprises from about 30% to about 99%, by weight of the non-lathering structured aqueous phase, of water. The non-lathering structured aqueous phase generally comprises more than about 50%, preferably more than about 60%, even more preferably more than about 70%, still more preferably more than about 80%, by weight of the non-lathering structured aqueous phase, of water.

The non-lathering structured aqueous phase will typically have a pH of from about 5 to about 8, more preferably about 7. The non-lathering structured aqueous phase can optionally comprise a pH regulator to facilitate the proper pH range.

The non-lathering structured aqueous phase can have a net cationic charge, net anionic charge, or neutral charge. In a preferred embodiment, the non-lathering structured aqueous phase has a net anionic charge.

The non-lathering structured aqueous phase of the present compositions can further comprise optional ingredients such as those described hereinafter. Preferred optional ingredients for the non-lathering structured aqueous phase include pigments, pH regulators, and preservatives. In one embodiment, the non-lathering structured aqueous phase comprises a water structurant (e.g. acrylates/vinyl isodecanoate crosspolymer), water, a pH regulator (e.g. triethanolamine), and a preservative (e.g. 1,3-dimethylol-5,5-dimethylhydantoin (“DMDMH” available from Lonza under the trade name GLYDANT®)).

A) Water Structurant

The non-lathering structured aqueous phase of the present invention comprises from about 0.1% to about 30%, preferably from about 0.5% to about 20%, more preferably from about 0.5% to about 10%, and even more preferably from about 0.5% to about 5%, by weight of the non-lathering structured aqueous phase, of a water structurant.

The water structurant is typically selected from the group consisting of inorganic water structurants, charged polymeric water structurants, water soluble polymeric structurants, associative water structurants, and mixtures thereof.

Non-limiting examples of inorganic water structurants for use in the personal cleansing composition include silicas, clays such as a synthetic silicates (Laponite XLG and Laponite XLS from Southern Clay), or mixtures thereof.

Non-limiting examples of charged polymeric water structurants for use in the personal cleansing composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), or mixtures thereof.

Non-limiting examples of water soluble polymeric structurants for use in the personal cleansing composition include cellulosic gel, hydroxypropyl starch phosphate (Structured XL from National Starch), polyvinyl alcohol, or mixtures thereof.

Nonlimiting examples of associative water structurants for use in the personal cleansing composition include xanthum gum, gellum gum, pectin, alginate, or mixtures thereof.

Particle

The multi-phase personal care composition of the present invention can comprise a particle. Water insoluble solid particle of various shapes and densities is useful. In a preferred embodiment, the particle tends to have a spherical, an oval, an irregular, or any other shape in which the ratio of the largest dimension to the smallest dimension (defined as the Aspect Ratio) is less than about 10. More preferably, the Aspect Ratio of the particle is less than about 8, still more preferably the Aspect Ratio of the particle is less than about 5.

The particle of the present invention has a particle size (volume average based on the particle size measurement described hereafter) of less than about 10,000 um, preferably less than about 1,000 um, more preferably less than 100 μM, even more preferably less than about 80 μM,.

The particle of the present invention preferably has a particle size of greater than about 0.1 μm, preferably a particle size of greater than about 0.5 μm, more preferably, a particle size greater than about 1 μm, still more preferably a particle size greater than about 2 μm, even more preferably a particle size greater than about 3 μm, and still even more preferably a particle size greater than about 4 μm.

The particle has a diameter from about 1 μm to about 70 μm, more preferably from about 2 μm to about 65 μm, and even more preferably from about 2 μm to about 60 μm in diameter.

The multi-phase personal care composition of the present invention comprises the particle at a cosmetically efficacious level. Preferably, the particles are present from at least about 0.1% by weight of the composition, more preferably at least about 0.2% by weight of composition, even more preferably at least about 0.5%, still more preferably at least about 1%, and even still more preferably at least 2% by weight of composition. In the multi-phase personal care composition of the present invention, preferably the particles comprises no more than about 50% by weight of composition, more preferably no more than about 30%, still more preferably no more than about 20%, and even more preferably no more than about 10% by weight of composition.

Preferably, the particle will also have physical properties which are not significantly affected by typical processing of the composition. Preferably, a particle having a melting point greater than about 70° C. is used, more preferably having a melting point greater than about 80° C., and even more preferably having a melting point of greater than about 95° C. is used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Non limiting examples of natural particles include various precipitated silica particles in hydrophilic and hydrophobic forms available from Degussa-Huls under the trade name Sipernet. Precipitated™, hydrophobic, synthetic amorphous silica, available from Degussa under the trade name Sipernet D11™ is a preferred particle. Snowtex colloidal silica particles available from Nissan Chemical America Corporation.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of inorganic pigments include iron oxides, ferric ammonium ferrocyanide, manganese violet, ultramarine blue, Chrome oxide, and Chromoxide Green (from Sun Chemical).

Non limiting examples of hybrid particles include Ganzpearl GSC-30SR (Sericite & crosslinked polystyrene hybrid powder), and SM-1000, SM-200 (mica and silica hybrid powder available from Presperse).

Exfoliant Particle

The exfoliant particle is selected from the group consisting of polyethylene, microcryatalline wax, jojoba esters, amourphors silica, talc, tracalcium orthophosphate, or blends thereof, and the like. The exfoliant particle has a particle size dimension along the major axis of the particle of from about 100 microns to about 600 microns, preferably from about 100 microns to about 300 microns. The exfoliant particle has a hardness of less than about 4 Mohs, preferably less than about 3 Mohs. The hardness as so measured is a criterion of the resistance of a particular material to crushing. It is known as being a fairly good indication of the abrasive character of a particulate ingredient. Examples of materials arranged in increasing order of hardness according to the Moh scale are as follows: h(hardness)-1:talc; h-2: gypsum, rock salt, crystalline salt in general, barytes, chalk, brimstone; h-4: fluorite, soft phosphate, magnesite, limestone; h-5: apatite, hard phosphate, hard limestone, chromite, bauxite; h-6: feldspar, ilmenite, hornblendes; h-7: quartz, granite; h-8: topaz; h-9: corrundum, emery; and h-10: diamond.

Preferably, the exfoliant particle has a color distinct from the cleansing base. The exfoliant particle is preferably present at a level of less than about 10%, preferably less than about 5%, by wt of the composition.

Shiny Particles

The multi-phase personal care compositions of the present invention can comprise a shiny particle in at least one phase of the multi-phase personal care composition. Nonlimiting examples of shiny particles include the following: interference pigment, multi-layered pigment, metallic particle, solid and liquid crystals, or combinations thereof.

An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer. The interference pigments of the multi-phased personal care compositions preferably comprises no more than about 20 weight percent of the composition, more preferably no more than about 10 weight percent, even more preferably no more than about 7 weight percent, and still more preferably no more than about 5 weight percent of the multi-phased personal care composition. The interference pigment of the multi-phased personal care composition preferably comprises at least about 0.1 weight percent of the multi-phased personal care composition, more preferably at least about 0.2 weight percent, even more preferably at least about 0.5 weight percent, and still more preferably at least about lweight percent by weight of the composition. When pigment is applied and rinsed as described in the Pigment Deposition Tape Strip Method as described in copending application Ser. No. 60/469,075 filed on May 8, 2003, the deposited pigment on the skin is preferably at least 0.5 μg/cm², more preferably at least 1 μg/cm², and even more preferably at least 5 μg/cm².

The interference pigments of the present invention are platelet particulates. The platelet particulates of the multi-phased personal care compositions preferably have a thickness of no more than about 5 μm, more preferably no more than about 2 μm, still more preferably no more than about 1 μm. The platelet particulates of the multi-phased personal care composition preferably have a thickness of at least about 0.02 μm, more preferably at least about 0.05 μm, even more preferably at least about 0.1 μm, and still more preferably at least about 0.2 μm.

The particle size determines the opacity and luster. The particle size is determined by measuring the diameter thickness of the particulate material. The term “diameter” as used herein, means the largest distance across the major axis of the particulate material. Diameter can be determined by any suitable method known in the art, such as particle size analyzer Mastersizer 2000 manufactured by Malvern Instruments. The interference pigment of the multi-phased personal care compositions preferably have an average diameter not greater than about 200 μm, more preferably not greater than 100 μm, even more preferably not greater than about 80 μm, still more preferably not greater than than about 60 μm. The interference pigment of the multi-phased personal care compositions preferably have a diameter of at least about 0.1 μm, more preferably at least about 1.0 μm, even more preferably at least about 2.0 μm, and still more preferably at least about 5.0 μm.

The interference pigment of the multi-phased personal care compositions comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, zeolite, boron nitride, oxychloride, silica flake, glass flake, ceramics, titanium dioxide, CaSO₄, CaCO₃, BaSO₄, borosilicate and mixtures thereof, preferably mica, cellulose acetate, PTFE, modified starch, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃ SnO₂. For the multiple layer structures, the thin films can be consisted of all high refractive index materials or alternation of thin films with high and low RI materials with the high RI film as the top layer.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine. It was found that the color of the interference pigment in addition to its brightness had a significant influence on human perception of skin tone. In general, preferred colors are silver, gold, red, green and mixtures thereof. In one preferred embodiment the human perception of skin tone is a whitening of ones skin tone.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®. In an embodiment of the present invention the interference pigment surface is either hydrophobic or has been hydrophobically modified. The Particle Contact Angle Test as described in copending application Ser. No.60/469,075 filed on May 8, 2003 is used to determine contact angle of interference pigments. The greater the contact angle, the greater the hydrophobicity of the interference pigment. The interference pigment of the present invention possess a contact angle of at least 60 degrees, more preferably greater than 80 degrees, even more preferably greater than 100 degrees, still more preferably greater than 100 degrees. The hydrophobically modified interference pigment or HMIP allows for the entrapment of the HMIP within the phases and greater deposition of the 1{I. Preferably the ratio of FMIP to a phase is 1:1 to about 1:70, more preferably 1:2 to about 1:50, still more preferably 1:3 to about 1:40 and most preferably 1:7 to about 1:35.

In an embodiment of the present invention the HMIP's are preferably entrapped within the benefit phase. This necessitates that the benefit phase particle size is generally larger than the HMIP. In a preferred embodiment of the invention, the benefit phase particles contain only a small number of HMIPs per benefit particles. Preferably this is less than 20, more preferably less than 10, most preferably less than 5. These parameters, the relative size of the benefit droplets to the HMIP and the approximate number of HMIP particles per benefit particles, can be determined by using visual inspection with light microscopy.

The HMIP and the benefit phase can be mixed into the composition via a premix or separately. For the case of separate addition, the hydrophobic pigments partition into the benefit phase during the processing of the formulation. The HMIP of the present invention preferably has a hydrophobic coating comprising no more than about 20 weight percent of the total particle weight, more preferably no more than about 15 weight percent, even more preferably no more than about 10 weight percent. The HMIP of the present invention preferably has a hydrophobic coating comprising at least about 0.1 weight percent of the total particle weight, more preferably at least about 0.5 weight percent, even more preferably at least about 1 weight percent. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

Skin Lightening Agents

The multi-phase personal care composition of the present invention can comprise a skin lightening agent. These skin lightening agent are preferably present at from about 0.0001% to about 20%, more preferably from about 0.05% to about 5%, even more preferably from about 0.1% to about 2%, by weight of the composition. Suitable skin lightening agents include those known in the art, including kojic acid, arbutin, tranexamic acid, ascorbic acid and derivatives thereof (e.g., magnesium ascorbyl phosphate or sodium ascorbyl phosphate, ascorbyl glucoside, and the like). Other skin lightening materials suitable for use herein include Actiwhite® (Cognis), Emblica® (Rona), Azeloglicina (Sinerga), Sepiwhite, hexamidine, sugar amines, (e.g., N-acetyl glucosamine), phytosterols (e.g. one or more sitosterol, stigmasterol, campesterol, brassicasterol, etc.), and extracts (e.g. mulberry extract).

Beads

The multi-phase personal care composition of the present invention can comprise beads. These beads are preferably present at from about 0.01% to about 10%, more preferably from about 0.1% to about 5%, even more preferably from about 0.5% to about 3%, by weight of the composition. The beads may be any color. The beads may be located in one phase or multiple phase of the of the multi-phase personal care composition. Beads may be used for signals for whitening, moisturizing, anti-aging, cleansing, exfoliation, scent bloom, scent longevity, and carriers for optional ingredients listed herein. Suitable beads include those known in the art, including soft and hard beads. Suitable examples of soft beads include unispheres, made by Induchem, Unispheres NT-2806 (Pink). Suitable examples of hard beads include oxidized polyethylene, preferably those made by Accutech.

Optional Ingredients

A variety of suitable optional ingredients can be employed in the multi-phase personal care composition. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional materials can be used in any aspect of the compositions of the present invention, including each phase as described herein.

Non-limiting optional ingredients include humectants and solutes. A variety of humectants and solutes can be employed and can be present at a level of from about 0.1% to about 50%, preferably from about 0.5% to about 35%, and more preferably from about 2% to about 20%, by weight of the personal care composition. A preferred humectant is glycerin.

A preferred water soluble, organic material is selected from the group consisting of a polyol of the structure:
R1-O(CH₂—CR2HO)ₙH
where R1=H, C1-C4 alkyl; R2=H, CH₃ and n=1-200; C2-C10 alkane diols; guanidin glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)ₓNH_{y} where x=1-3; y=0-2, and x+y=3, and mixtures thereof. The most preferred polyols are selected from the group consisting of glycerine, polyoxypropylene(1) glycerol and polyoxypropylene(3) glycerol, sorbitol, butylene glycol, propylene glycol, sucrose, urea and triethanol amine.

Nonionic polyethylene/polypropylene glycol polymers are preferably used as skin conditioning agents. Polymers useful herein that are especially preferred are PEG-2M wherein x equals 2 and n has an average value of about 2,000 (PEG 2-M is also known as Polyox WSR® N-10 from Union Carbide and as PEG-2,000); PEG-5M wherein x equals 2 and n has an average value of about 5,000 (PEG 5-M is also known as Polyox WSR® 35 and Polyox WSR® N-80, both from Union Carbide and as PEG-5,000 and Polyethylene Glycol 200,000); PEG-7M wherein x equals 2 and n has an average value of about 7,000 (PEG 7-M is also known as Polyox WSR® (N-750 from Union Carbide); PEG-9M wherein x equals 2 and n has an average value of about 9,000 (PEG 9-M is also known as Polyox WSR® N-3333 from Union Carbide); PEG-14 M wherein x equals 2 and n has an average value of about 14,000 (PEG 14-M is also known as Polyox WSR-205 and Polyox WSR® N-3000 both from Union Carbide); and PEG-90M wherein x equals 2 and n has an average value of about 90,000. (PEG-90M is also known as Polyox WSR®-301 from Union Carbide.)

Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Viscosity Method

The Wells-Brookfield Cone/Plate Model DV-II+ Viscometer can be used to determine the viscosity of the non-lathering structured aqueous phase and the lathering cleansing phase herein. The determination is performed at 25° C. with the 2.4 cm 2° cone measuring system with a gap of 0.013 mm between the two small pins on the respective cone and plate. The measurement is performed by injecting 0.5 ml of the sample, and then, rotating the cone at a set speed of 1 rpm. The resistance to the rotation of the cone produces a torque that is proportional to the shear stress of the liquid sample. The amount of torque is read 2 minutes after loading the sample and computed by the viscometer into absolute centipoise units (mPa*s) based on the geometric constant of the cone, the rate of rotation, and the stress related torque.

Yield Point Method

A TA Instruments AR2000 Controlled Stress Rheometer can be used to determine the Yield Point of the non-lathering structured aqueous phase or the lathering cleansing phase. For purpose herein, the Yield Point is the amount of stress required to produce a strain of 1% on the liquid non-lathering structured aqueous phase or the lathering cleansing phase. The determination is performed at 25° C. with the 4 cm diameter parallel plate measuring system and a 1 mm gap. The determination is performed via the programmed application of a shear stress (typically from about 0.1 Pa to about 500 Pa) over a time interval of 5 minutes. It is this amount of stress that results in a deformation of the sample, a shear stress vs. strain curve can be created. From this curve, the Yield Point of the liquid non-lathering structured aqueous phase can be determined. The liquid non-lathering structured aqueous phase or the lathering cleansing phase are measured either prior to combining in the composition, or after combining in the composition by separating the compositions by suitable physical separation means, such as centrifugation, pipetting, cutting away mechanically, rinsing, filtering, or other separation means.

Correlated Haze Index Method

The Macbeth Color Measurement Sytem-Gretag Macbeth Model 7000 with sphere geometry optical head is used to perform the Correlated Haze Index Method. The instrument needs to be calibrated on both reflectance and transmission modes. Both of these calibrations are used to obtain the Correlated Haze Index.

To prepare the sample, the composition is centrifuged at 3000 rpm for about 3 minutes to remove any air bubbles that may be present. Then, slowly pour the composition into an optical cell to avoid air entrapment. If the air entrapment occurs, allow the sample to sit for 30 minutes at room temperature to de-aerate. If air bubbles persist, first empty the cell, then clean and dry the cell and then refill as before. Remove any composition spilled on the outside surface of the cell by for example wiping. The sample of the composition must be within 2 C of the original calibration temperature.

Once the sample is prepared, the instrument should be on traditional Lab setting, using C Illuminate, 2 degree observer angle and no averaging. Next configure the instrument setting to CRIOLL setting. This is done by changing the specular component to included, the UV to excluded, and the measurement mode to reflectance. These changes are made without any sample cell holder inside the instrument. Next, place a large sample cell holder without sample inside the instrument and calibrate the instrument according to on screen prompts. Switch the measurement mode to transmission, then the instrument will show BTIOLL setting. Calibrate the instruments by following onscreen prompts.

Next, switch the instrument to measurement mode, Correlated Haze. The instrument setting will now be XHIOLL. Calibrate the instrument by following the onscreen prompts. The new instrument setting will be CHIOLL. The operator then clicks the indices icon on the toolbar to bring up the display that shows Correlated Haze results. Run an empty cell as the standard.

Fill the optical cell with the sample of the composition to be analyzed, making sure there is no air entrapment. Run as a trial and report percent Correlated Haze results. The calibration of the instrument must be performed at least every 8 hours.

Particle Size Measurement Method:

The particle size measurement method is typical of those known in the art, and utilizes a standard Nikon optical microscope, with standard transmitted light using ×10 objective. To aid accuracy, a Lucia G software (by Nikon) is used with the following procedure. The first step of analysis requires the user to scan and select a field that is representative of the bulk—this typically requires multiple preparations for accuracy. The observed image is transmitted via JVC video camera to a standard monitor and each particle is measured by using the standard Measure macro; namely, clicking on each side of the particle—hence measuring a diameter. To account for none spherical particles, the ‘diameter’ is always assessed horizontally across the monitor. By measuring in one plane, the technique automatically compensates for non spherical geometry and due to the large number of particles measured results in an equivalent average diameter. Although equivalent diameters may be determined by measuring the major and minor axes and calculating equivalent diameter via aspect ratio equations, the above technique provides equally accurate results.

Since it is typical human nature to count the largest particles first and thus to ensure that all particles are counted and measured, a small (typically using an erasable pen) dot should be placed on the monitor over each counted particle. The count procedure is continued until every single visible particle is counted within the field. In the case of a very small particle size distribution, this may result in over 400 counts. In the case of larger particle sizes, one might expect approximately 100 counts per field, however in such cases additional fields would be selected to ensure at least 200 separate particles are counted. In summary, in all cases at least 200 separate particles should be measured and in all cases all particles (in practice the upper limit being 400-500) in one field are counted. On average, across all the examples sighted herein, about 300 particles would be measured per sample. Analysis can be (standard volume average calculated by hand to demonstrate the technique) or, more typically, using the standard Measure macro that automatically sorts the data reporting a volume average (assuming a spherical geometry based on the diameter measured above).

Method Of Use

The multi-phase personal cleansing compositions of the present invention are preferably applied topically to the desired area of the skin or hair in an amount sufficient to provide effective delivery of the skin cleansing agent, hydrophobic material, and particles to-the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water.

The present invention is therefore also directed to methods of cleansing the skin through the above-described application of the compositions of the present invention. The methods of the present invention are also directed to a method of providing effective delivery of the desired skin active agent, and the resulting benefits from such effective delivery as described herein, to the applied surface through the above-described application of the compositions of the present invention.

Method Of Manufacture

The multi-phase personal cleansing compositions of the present invention may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples.

If the multi-phase personal cleansing compositions contain patterns of varying colors it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Each of the examples below are of multi-phase personal care compositions comprising 50%, by weight of the personal care composition, of a first phase and 50%, by weight of the multi-phase personal care composition, of a second phase. The amount of each component in a particular phase is provided as a weight percent based on the weight of the particular phase that contains the component.

### Examples 1-3

The following examples described are non-limiting examples of multi-phase personal care compositions.

| | Ex 1 | Ex 2 | Ex 3 |
|---|---|---|---|
| Ingredient | wt % | Wt % | Wt % |
| I. First Phase Composition | | | |
| Miracare SLB-365 (from Rhodia) | 47.4 | 47.4 | 47.4 |
| (Sodium Trideceth Sulfate, Sodium | | | |
| Lauramphoacetate, Cocamide MEA) | | | |
| Guar Hydroxypropyltrimonium Chloride (N- | 0.7 | 0.7 | 0.7 |
| Hance 3196 from Aqualon) | | | |
| PEG 90M (Polyox WSR 301 from Dow | 0.2 | 0.2 | 0.2 |
| Chemical) | | | |
| Sodium Chloride | 3.5 | 3.5 | 3.5 |
| Preservatives | 0.84 | 0.84 | 0.84 |
| Citric Acid | 0.4 | 0.4 | 0.4 |
| Perfume | 2.0 | 2.0 | 2.0 |
| Expancel 091 DE 40 d30 | 0.5 | 0.5 | 0.5 |
| (from Expancel, Inc.) | | | |
| Water | Q.S. | Q.S. | Q.S. |
| (pH) | (6.0) | (6.0) | (6.0) |
| II. Second Phase Composition | | | |
| Petrolatum (Superwhite Protopet from | 59.95 | — | — |
| WITCO) | | | |
| Petrolatum (G2218 Petrolatum | — | 69.95 | 69.45 |
| from WITCO) | | | |
| Mineral Oil (Hydrobrite 1000 PO White MO | 40.0392 | 30.0392 | 30.0392 |
| from WITCO) | | | |
| Chromoxide Green (from Sun Chemical) | 0.0073 | 0.0073 | 0.0073 |
| Green #6 Oil Soluble Dye | 0.0035 | 0.0035 | 0.0035 |
| Titanium Dioxide | — | — | 0.5 |
| (SAT-T-CR50 Silicone Treated from US | | | |
| Cosmetics) | | | |

The multi-phase personal care compositions described above can be prepared by conventional formulation and mixing techniques. Prepare the cleansing phase composition by first add citric acid into water at 1:3 ratio to form a citric acid premix. Disperse Polyox WSR 301 in glycerin at 1:3 ratio to form a Polyox premix. Disperse Polymer N-Hance in water at 1: 10 ratio to form a N-Hance premix. Then, add the following ingredients into the main mixing vessel in the following sequence: water, N-Hance premix, Polyox premix, citric acid premix, and Expancel. Start agitation of the mixing vessel. Then, add Miracare SLB-365, sodium chloride, sodium benzoate, Disodium EDTA, glydant, and perfume. Keep agitation until a homogenous solution forms.

The second phase can be prepared by adding petrolatum into a mixing vessel. Heat the vessel to 190° F. (88° C.). Then, add mineral oil, oil soluble dye, Chromoxide Green, and Titanium Dioxide. High shear the batch to ensure good particle dispersion. Keep agitating the batch and slowly cool down the batch to ambient temperature.

These phases can be combined by first placing the separate phases in separate storage tanks having a pump and a hose attached. The phases are then pumped in predetermined amounts (50% v/v Surfactant Phase and 50% v/v Benefit Phase) into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

### Examples 4-6

The following examples described are non-limiting examples of multi-phase personal care compositions.

| | Ex- | Ex- | Ex- |
|---|---|---|---|
| | ample 4 | ample 5 | ample 6 |
| Ingredient | wt % | wt % | wt % |
| I. Lathering Cleansing First Phase | | | |
| Composition | | | |
| Miracare SLB-365 (from Rhodia) | 47.4 | 47.4 | 47.4 |
| (Sodium Trideceth Sulfate, Sodium | | | |
| Lauramphoacetate, Cocamide MEA) | | | |
| Cocamide MEA | 3.0 | 3.0 | 3.0 |
| Guar Hydroxypropyltrimonium Chloride | 0.7 | 0.7 | 0.7 |
| (N-Hance 3196 from Aqualon) | | | |
| PEG 90M (Polyox WSR 301 from Dow | 0.2 | 0.2 | 0.2 |
| Chemical) | | | |
| Glycerin | 0.8 | 0.8 | 0.8 |
| Sodium Chloride | 3.5 | 3.5 | 3.5 |
| Disodium EDTA | 0.05 | 0.05 | 0.05 |
| Glydant | 0.67 | 0.67 | 0.67 |
| Citric Acid | 0.4 | 0.4 | 0.4 |
| Perfume | 2.0 | 2.0 | 2.0 |
| Water | Q.S. | Q.S. | Q.S. |
| (pH) | (6.0) | (6.0) | (6.0) |
| II. Non-Lathering Structured Aqueous | | | |
| Phase Composition | | | |
| Acrylates/Vinyl Isodecanoate Crosspolymer | 1.0 | 1.0 | 1.0 |
| (Stayblen 30 from 3V) | | | |
| Xanthan Gum (Keltrol CGT from CP Kelco) | 1.0 | 1.0 | 1.0 |
| Hydrobrite 1000 White Mineral Oil | 0.08 | — | 0.08 |
| FD&C Blue #1 Lake (from Sun Chemical) | 0.015 | — | 0.015 |
| Chromoxide Green (from Sun Chemical) | 0.005 | — | 0.005 |
| Unisperes NT-2806 Green Beads (from | — | 5 | — |
| Induchem) | | | |
| Titanium Dioxide | — | — | 0.5 |
| (SAT-T-CR50 Silicone Treated from US | | | |
| Cosmetics) | | | |
| Triethanolamine | 1.82 | 1.82 | 1.82 |
| Glydant | 0.37 | 0.37 | 0.37 |
| Sodium Chloride | 3 | 3 | 3 |
| Disodium EDTA | 0.05 | 0.05 | 0.05 |
| Water and Minors | Q.S. | Q.S. | Q.S. |
| (pH) | (6.0) | (6.0) | (6.0) |

The compositions described above can be prepared by conventional formulation and mixing techniques. The lathering cleansing phase composition can be prepared by forming the following premixes: adding citric acid into water at 1:1 ratio to form a citric acid premix, add polyox WSR-301 into glycerin at 1:3 ratio to form a polyox-glycerin premix, mix well using a high shear mixer. Then add the following ingredient in the main mixing vessel in the following sequence: water, N-Hance 3196, polyox premix, citric acid premix, disodium EDTA, and Miracare SLB-365. After mixing for 30 mins, begin heating the batch to 120 F. Add CMEA and mix until homogeneous. Then cool the batch to ambient temperature and add the following ingredients: sodium chloride, glydant, and perfume. Mix the batch for 60 mins. Check pH and adjust pH to 6.0 using citric acid or caustic solution if needed.

The non-lathering structured aqueous phase can be prepared by first preparing the Pigment-Mineral Oil premix by adding FD&C Blue #1 and Chromoxide Green in Hydrobrite 1000 White Mineral Oil at 1:4 ratio. High shear the premix. Then, slowly add Stabylen 30 into water in the main mixing vessel. Then, add Pigment-Mineral Oil premix, Titanium Dioxide and Xanthan Gum. High shear the mixture. Then, slowly add Triethanolamine, Sodium Chloride, Glydant, Disodium EDTA, and Unisperes (Green Beads) with agitation. Adjust pH to about 6.0 using citric acid or Triethanolamine.

The cleansing and non-lathering structured aqueous phases can be combined by first placing the separate phases in separate storage tanks having a pump and a hose attached. The phases are then pumped in predetermined amounts (e.g,, 50/50 v/v ratio) into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

### Examples 7-11

The following examples described are non-limiting examples of multi-phase compositions.

| | Ex 7 | Ex 8 | Ex 9 | Ex 10 | Ex 11 |
|---|---|---|---|---|---|
| Ingredient | wt % | wt % | wt % | wt % | wt % |
| I. First Phase Composition | | | | | |
| Miracare SLB-365 (from Rhodia) | 47.4 | 47.4 | 47.4 | 47.4 | 47.4 |
| (Sodium Trideceth Sulfate, Sodium | | | | | |
| Lauramphoacetate, Cocamide MEA) | | | | | |
| Guar Hydroxypropyltrimonium Chloride | 0.7 | 0.7 | 0.7 | 0.7 | 0.7 |
| (N-Hance 3196 from Aqualon) | | | | | |
| PEG 90M (Polyox WSR 301 from Dow | 0.2 | 0.2 | 0.2 | 0.2 | 0.2 |
| Chemical) | | | | | |
| Sodium Chloride | 3.5 | 3.5 | 3.5 | 3.5 | 3.5 |
| Preservatives | 0.84 | 0.84 | 0.84 | 0.84 | 0.84 |
| Citric Acid | 0.4 | 0.4 | 0.4 | 0.4 | 0.4 |
| Perfume | 2.0 | 2.0 | 2.0 | 2.0 | 2.0 |
| Expancel 091 DE 40 d30 (from Expancel, Inc.) | 0.4 | 0.4 | 0.4 | 0.4 | 0.4 |
| Water | Q.S. | Q.S. | Q.S. | Q.S. | Q.S. |
| (pH) | (6.0) | (6.0) | (6.0) | (6.0) | (6.0) |
| II. Second Phase Composition | | | | | |
| Petrolatum (Superwhite Protopet from | 57.0 | 58.5 | 57.75 | 58.5 | 57.0 |
| WITCO) | | | | | |
| Mineral Oil (Hydrobrite 1000 PO White | 38.0 | 39.0 | 38.5 | 39.0 | 38.0 |
| MO from WITCO) | | | | | |
| Mica/Titanium Dioxide/Tin | 1.25 | 1.875 | | | |
| Oxide/Triethoxy caprylylsilane | | | | | |
| (Kobopearl Interval Gold-11S2 from | | | | | |
| Kobo Products Inc.) | | | | | |
| Mica/Titanium Dioxide/Triethoxy | 2.50 | | | | |
| caprylylsilane (Kobopearl Stellar White- | | | | | |
| 11S2 from Kobo Products Inc.) | | | | | |
| Titanium | | | 1.875 | | |
| Dioxide/Mica/Silica/Dimethicone (SAT- | | | | | |
| Timiron Splendid Red from US | | | | | |
| Cosmetics) | | | | | |
| Mica/Titanium Dioxide/Triethoxy | 1.25 | 0.625 | | | |
| caprylylsilane/Iron Oxide/Tin Oxide | | | | | |
| (Kobopearl Vibrant Gold-11S2 from | | | | | |
| Kobo Products Inc.) | | | | | |
| Mica/Titanium Dioxide/Tin | | | | 1.75 | |
| Oxide/Triethoxy caprylylsilane | | | | | |
| (Kobopearl Interval Red-11S2 from | | | | | |
| Kobo Products Inc.) | | | | | |
| Mica/Mineral/Titanium Dioxide/Iron | | | | | 2.50 |
| Oxide/Lecithin (LT-Colorona Red Gold | | | | | |
| from US Cosmetics) | | | | | |
| Mica/Titanium Dioxide/Dimethicone | | | | | |
| (SAT-Timiron Super Green from US | | | | | |
| Cosmetics) | | | | | |
| Mica/Dimethicone (SA-M-M from US | | | | | |
| Cosmetics) | | | | | |
| Mica/Titanium Dioxide/Dimethicone | | | | | |
| (SAT-Flamenco Ultra Silk 2500 from | | | | | |
| US Cosmetics) | | | | | |
| Polyacrylate-4 (Helicone HC Maple | | | | | |
| from Kobo Products, Inc.) | | | | | |
| Mica/Titanium Dioxide/Dimethicone | | | 1.875 | 1.75 | 2.50 |
| (SAT-Timiron MP115 Starluster from | | | | | |
| US Cosmetics) | | | | | |

The compositions described above can be prepared by conventional formulation and mixing techniques. Prepare the first phase composition by first adding citric acid into water at 1:3 ratio to form a citric acid premix. Then, add the following ingredients into the main mixing vessel in the following sequence: water, Miracare SLB-365, sodium chloride, sodium benzoate,

Disodium EDTA, glydant. Start agitation of the main mixing vessel. In a separate mixing vessel, disperse polymers (Polyquaterium 10, Jaguar C-17, or N-Hance 3196) in water at 1:10 ratio and form a polymer premix. Add the completely dispersed polymer premix into the main mixing vessel with continuous agitation. Disperse Polyox WSR 301 in water and then add to the main mixing vessel. Then, add the rest of the water and perfume into the batch. Keep agitation until a homogenous solution forms.

The second phase can be prepared by adding petrolatum into a mixing vessel. Heat the vessel to 190° F. (88° C.). Then, add mineral oil and particles. High shear the batch to ensure good particle dispersion. Keep agitating the batch and slowly cool down the batch to ambient temperature.

These phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

### Examples 12-16

The following examples described are non-limiting examples of multi-phase compositions.

| | Ex 12 | Ex 13 | Ex 14 | Ex 15 | Ex 16 |
|---|---|---|---|---|---|
| Ingredient | wt % | wt % | wt % | wt % | wt % |
| I. First Phase Composition | | | | | |
| Miracare SLB-365 (from Rhodia) | 47.4 | 47.4 | 47.4 | 47.4 | 47.4 |
| (Sodium Trideceth Sulfate, Sodium | | | | | |
| Lauramphoacetate, Cocamide MEA) | | | | | |
| Guar Hydroxypropyltrimonium Chloride | 0.7 | 0.7 | 0.7 | 0.7 | 0.7 |
| (N-Hance 3196 from Aqualon) | | | | | |
| PEG 90M (Polyox WSR 301 from Dow | 0.2 | 0.2 | 0.2 | 0.2 | 0.2 |
| Chemical) | | | | | |
| Sodium Chloride | 3.5 | 3.5 | 3.5 | 3.5 | 3.5 |
| Preservatives | 0.84 | 0.84 | 0.84 | 0.84 | 0.84 |
| Citric Acid | 0.4 | 0.4 | 0.4 | 0.4 | 0.4 |
| Perfume | 2.0 | 2.0 | 2.0 | 2.0 | 2.0 |
| Expancel 091 DE 40 d30 (from Expancel, Inc.) | 0.4 | 0.4 | 0.4 | 0.4 | 0.4 |
| Water | Q.S. | Q.S. | Q.S. | Q.S. | Q.S. |
| (pH) | (6.0) | (6.0) | (6.0) | (6.0) | (6.0) |
| II. Second Phase Composition | | | | | |
| Petrolatum (Superwhite Protopet from | 57.75 | 57.0 | 58.5 | 57.0 | 57.0 |
| WITCO) | | | | | |
| Mineral Oil (Hydrobrite 1000 PO White | 38.50 | 38.0 | 39.0 | 38.0 | 38.0 |
| MO from WITCO) | | | | | |
| Mica/Titanium Dioxide/Tin | | | | | 1.875 |
| Oxide/Triethoxy caprylylsilane | | | | | |
| (Kobopearl Interval Gold-11S2 from | | | | | |
| Kobo Products Inc.) | | | | | |
| Mica/Titanium Dioxide/Triethoxy | | | | | |
| caprylylsilane (Kobopearl Stellar White- | | | | | |
| 11S2 from Kobo Products Inc.) | | | | | |
| Titanium | | | | | |
| Dioxide/Mica/Silica/Dimethicone (SAT- | | | | | |
| Timiron Splendid Red from US | | | | | |
| Cosmetics) | | | | | |
| Mica/Titanium Dioxide/Triethoxy | | | | | 3.125 |
| caprylylsilane/Iron Oxide/Tin Oxide | | | | | |
| (Kobopearl Vibrant Gold-11S2 from | | | | | |
| Kobo Products Inc.) | | | | | |
| Mica/Titanium Dioxide/Tin | | | | | |
| Oxide/Triethoxy caprylylsilane | | | | | |
| (Kobopearl Interval Red-11S2 from | | | | | |
| Kobo Products Inc.) | | | | | |
| Mica/Mineral/Titanium Dioxide/Iron | | | | | |
| Oxide/Lecithin (LT-Colorona Red Gold | | | | | |
| from US Cosmetics) | | | | | |
| Mica/Titanium Dioxide/Dimethicone | 2.813 | | | | |
| (SAT-Timiron Super Green from US | | | | | |
| Cosmetics) | | | | | |
| Mica/Dimethicone (SA-M-M from US | | 5.0 | | | |
| Cosmetics) | | | | | |
| Mica/Titanium Dioxide/Dimethicone | | | 2.50 | | |
| (SAT-Flamenco Ultra Silk 2500 from | | | | | |
| US Cosmetics) | | | | | |
| Polyacrylate-4 (Helicone HC Maple | | | | 5.0 | |
| from Kobo Products, Inc.) | | | | | |
| Mica/Titanium Dioxide/Dimethicone | .938 | | | | |
| (SAT-Timiron MP115 Starluster from | | | | | |
| US Cosmetics) | | | | | |

The compositions described above can be prepared by conventional formulation and mixing techniques. Prepare the first phase composition by first adding citric acid into water at 1:3 ratio to form a citric acid premix. Then, add the following ingredients into the main mixing vessel in the following sequence: water, Miracare SLB-365, sodium chloride, sodium benzoate, Disodium EDTA, glydant. Start agitation of the main mixing vessel. In a separate mixing vessel, disperse polymers (Polyquaterium 10, Jaguar C-17, or N-Hance 3196) in water at 1:10 ratio and form a polymer premix. Add the completely dispersed polymer premix into the main mixing vessel with continuous agitation. Disperse Polyox WSR 301 in waterl and then add to the main mixing vessel. Then, add the rest of the water and perfume into the batch. Keep agitation until a homogenous solution forms.

The second phase can be prepared by adding petrolatum into a mixing vessel. Heat the vessel to 190° F. (88° C.). Then, add mineral oil and particles. High shear the batch to ensure good particle dispersion. Keep agitating the batch and slowly cool down the batch to ambient temperature.

These phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

### Examples 17-19

The following examples described are non-limiting examples of multi-phase compositions.

| | Ex. 17 | Ex. 18 | Ex. 19 |
|---|---|---|---|
| Ingredient | wt % | wt % | Wt % |
| I. First Phase Composition | | | |
| Ammonium Laureth-3 Sulfate | 3.0 | 3.0 | 3.0 |
| Sodium Lauroamphoacetate | 16.7 | 16.7 | 16.7 |
| (Miranol L-32 Ultra from Rhodia) | | | |
| Ammonium Lauryl Sulfate | 1.0 | 1.0 | 1.0 |
| Lauric Acid | 0.9 | 0.9 | 0.9 |
| Trihydroxystearin (Thixcin R) | 2.0 | 2.0 | 2.0 |
| Guar Hydroxypropyltrimonium Chloride | 0.17 | 0.75 | 0.75 |
| (N-Hance 3196 from Aqualon) | | | |
| Guar Hydroxypropyltrimonium Chloride | 0.58 | — | — |
| (Jaguar C-17 from Rhodia) | | | |
| Polyquaterium 10 | 0.45 | — | — |
| (UCARE polymer JR-30M from Amerchol) | | | |
| Polymethacrylamidopropyltrimonium | — | 0.24 | — |
| Chloride (Polycare 133 from Rhodia) | | | |
| Polyquaternium-39 | — | 0.81 | — |
| (Merqurt Plus 3300 from Calgon) | | | |
| PEG 90M (Polyox WSR 301 from Union | 0.25 | — | — |
| Carbide) | | | |
| PEG-14M (Polyox WSR N-3000 H from | 0.45 | 2.45 | 2.45 |
| Union Carbide) | | | |
| Linoleamidoprypyl PG-Dimonium Chloride | — | 1.0 | 4.0 |
| Phosphate Dimethicone | | | |
| (Monasil PLN from Uniqema) | | | |
| Glycerin | 1.4 | 4.9 | 4.9 |
| Sodium Chloride | 0.3 | 0.3 | 0.3 |
| Sodium Benzoate | 0.25 | 0.25 | 0.25 |
| Disodium EDTA | 0.13 | 0.13 | 0.13 |
| Glydant | 0.37 | 0.37 | 0.37 |
| Citric Acid | 1.6 | 0.95 | 0.95 |
| Titanium Dioxide | 0.5 | 0.5 | 0.5 |
| Perfume | 0.5 | 0.5 | 0.5 |
| Water | Q.S. | Q.S. | Q.S. |
| II. Second Phase Composition | | | |
| Petrolatum (SuperWhite Protopet from | 60 | 60 | 60 |
| WITCO) | | | |
| Mineral Oil (Hydrobrite 1000 White MO | 38 | 28 | 28 |
| from WITCO) | | | |
| Tospearl 2000 (from GE) | — | 10 | — |
| Dry-Flo AF (from National Starch) | — | — | 10 |
| Colorona Magenta Cosmetic Pigment (from | 2 | 2 | 2 |
| Rona). | | | |

The first phase compositions described above can be prepared by conventional formulation and mixing techniques. The first phase composition of Example 17 can be prepared by first creating the following premixes: citric acid in water premix at 1:3 ratio, Guar polymer premix with Jaguar C-17 and N-Hance 3196 in water at 1:10 ratio, UCARE premix with JR-30M in water at about 1:30 ratio, and Polyox premix with PEG-90M and PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, UCARE premix, Polyox Premix, and the rest of water. Heat the vessel with agitation until it reaches 190° F. (88° C.). Let it mix for about 10 min. Cool the batch with a cold water bath with slow agitation until it reaches 110° F. (43° C.). Add the following ingredients: Glydant, perfume, Titanium Dioxide. Keep mixing until a homogeneous solution forms.

The composition of Example 18 can be prepared by first creating the following premixes:

citric acid in water premix at 1:3 ratio, Guar polymer premix with N-Hance 3196 in water at 1:10 ratio, and Polyox premix with PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, Polyox Premix, Polycare 133, Merquat Plus 3300, Monosil PLN, and the rest of water. Heat the vessel with agitation until it reaches 190° F. (88° C.). Let it mix for about 10 min. Cool the batch with a cold water bath with slow agitation until it reaches 110° F. (43° C.). Add the following ingredients: Glydant, perfume, Titanium Dioxide. Keep mixing until a homogeneous solution forms.

The composition of Example 19 can be prepared by first creating the following premixes: citric acid in water premix at 1:3 ratio, Guar polymer premix with N-Hance 3196 in water at 1:10 ratio, and Polyox premix with PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, Polyox Premix, Monasil PLN, and the rest of water. Heat the vessel with agitation until it reaches 190° F. (88° C.). Let it mix for about 10 min. Cool the batch with a cold water bath with slow agitation until it reaches 110° F. (43° C.). Add the following ingredients: Glydant, perfume, Titanium Dioxide. Keep mixing until a homogeneous solution forms.

The second phase can be prepared by adding petrolatum into a mixing vessel. Heat the vessel to 190° F. (88° C.). Then, add mineral oil, cosmetic pigment, and Dry-Flo AF or Tospearl with agitation. Let the vessel cool down with slow agitation.

These phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

### Examples 20-22

The following examples are non-limiting examples of multi-phase compositions of the present invention.

| | Ex. 20 | Ex. 21 | Ex. 22 |
|---|---|---|---|
| Ingredient | wt % | wt % | wt % |
| I. First Phase Composition | | | |
| Miracare SLB-365 (from Rhodia) | 47.4 | 47.4 | 47.4 |
| (Sodium Trideceth Sulfate, Sodium | | | |
| Lauramphoacetate, Cocamide MEA) | | | |
| Sodium Chloride | 3.5 | 3.5 | 3.5 |
| Disodium EDTA | 0.05 | 0.05 | 0.05 |
| Glydant | 0.67 | 0.67 | 0.67 |
| Citric Acid | 0.4 | 0.4 | 0.4 |
| Perfume | 2.0 | 2.0 | 2.0 |
| Water | Q.S. | Q.S. | Q.S. |
| (pH) | (6.0) | (6.0) | (6.0) |
| II. Second Phase Composition | | | |
| Acrylates/Vinyl Isodecanoate Crosspolymer | 0.8 | 1.0 | 1.2 |
| (Stayblen 30 from 3V) | | | |
| Triethanolamine | 0.8 | 1.0 | 1.2 |
| Glydant | 0.37 | 0.37 | 0.37 |
| Unispheres NT-2806 (Pink) | 5 | 5 | 5 |
| (Mannitol, Cellulose, | | | |
| Hydroxypropylcelllulose from Induchem) | | | |
| Water and Minors | 93.03 | 92.63 | 92.23 |
| (pH) | (7.0) | (7.0) | (7.0) |

The compositions described above can be prepared by conventional formulation and mixing techniques. The first phase composition is prepared by first add citric acid into water at 1:3 ratio to form a citric acid premix. Then, add the following ingredients into the main mixing vessel in the following sequence: water, Miracare SLB-354, sodium chloride, sodium benzoate, Disodium EDTA, glydant. Start agitation of the main mixing vessel. In a separate mixing vessel, disperse polymers (N-Hance 3196) in water at 1:10 ratio and form a polymer premix. Add the completely dispersed polymer premix into the main mixing vessel with continuous agitation. Disperse Polyox WSR 301 in waterl and then add to the main mixing vessel. Then, add the rest of the water and perfume into the batch. Keep agitation until a homogenous solution forms.

The second phase can be prepared by slowly adding Stabylen 30 into water in a mixing vessel. Then, add Triethanolamine, Glydant, Unisphere NT-2806 (Pink) with agitation. Mix until homogeneous.

These phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A multi-phase personal care composition comprising at least two visually distinct phases;
wherein the phases form a pattern;
wherein at least one phase comprises a particle;
wherein said particle is present at a cosmetically efficacious level; and
wherein said phases are packaged in physical contact with one another and maintain stability.

2. The multi-phase personal care composition of claim 1, wherein said phases are selected from the group consisting of a cleansing phase, a benefit phase, a non-lathering structured aqueous phase and combinations thereof.

3. The multi-phase personal care composition of claim 1, wherein said pattern is selected from the group consisting of striped, geometric, marbled and combinations thereof.

4. The multi-phase personal care composition of claim 3, wherein said pattern is striped, said striped pattern having a size at least from about 0.1 mm in width and about 10 mm in length.

5. The multi-phase personal care composition of claim 1, comprising at least about 0.1% by weight of the composition, of said particle.

6. The multi-phase personal care composition of claim 1, comprising at least about 0.2% by weight of composition, of said particle.

7. The multi-phase personal care composition of claim 1, wherein said particle has a particle size of less than about 10 mm.

8. The multi-phase personal care composition of claim 1, wherein said particle has a diameter from about 1 μm to about 70 μm.

9. The multi-phase personal care composition of claim 1, wherein said particle is selected from the group consisting of natural, synthetic, semi-synthetic, hybrid and combinations thereof.

10. The multi-phase personal care composition of claim 1, wherein said particle is selected from the group consisting of exfoliant particle, shiny particle, and combinations thereof.

11. The multi-phase personal care composition of claim 10, wherein said shiny particle is an interference pigment; wherein said interference pigment is a hydrophobically modified interference pigment; and wherein said composition comprises at least about 0.1% by weight of said composition of said hydrophobically modified interference pigment.

12. The multi-phase personal care composition of claim 11, wherein said composition deposits at least about 0.5 μg/cm² of said hydrophobically modified interference pigments on the skin.

13. The multi-phase personal care composition of claim 1, wherein said composition further comprises a bead.

14. The multi-phase personal care composition of claim 1, wherein said composition further comprises a skin lightening agent.

15. The multi-phase personal cleansing composition of claim 2, wherein at least one phase is said cleansing phase comprising:
(i) at least one anionic surfactant;
(ii) at least one electrolyte;
(iii) at least one alkanolamide;
(iv) optionally additional conventional surfactants; and
(v) water;
wherein the cleansing phase is non-Newtonian shear thinning; and
the cleansing phase has a viscosity of equal to or greater than about 3000 cps.

16. The multi-phase -personal care composition of claim 15, wherein the cleansing phase comprises from about 1% to about 50% by weight of said cleansing phase, of a surfactant.

17. The multi-phase personal care composition of claim 15, wherein said electrolyte comprises
i) an anion selected from the group consisting of phosphate, chloride, sulfate, citrate and mixtures thereof, and
ii) a cation selected from the group consisting of sodium, ammonium, potassium, magnesium and mixtures thereof; and wherein the electrolyte is present at a level from about 0.1% to about 15%, by weight of said cleansing phase.

18. The multi-phase personal care composition of claim 15, wherein the cleansing phase additionally comprises a lamellar structurant; wherein said lamellar structurant is selected from the group consisting of fatty acids, fatty esters, trihydroxystearin, fatty alcohols, and mixtures thereof.

19. The multi-phased personal care composition of claim 15, further comprising a cationic deposition polymer.

20. A multi-phase personal care composition comprising; at least two visually distinct phases;
wherein said phases form a pattern;
wherein at least one phase comprises a particle;
wherein a ratio of a first phase to a second phase is about 90:10 to about 10:90; and
wherein said phases are packaged in physical contact with one another and maintain stability.

21. The multi-phase personal care composition of claim 20, wherein said phases are selected from the group consisting of a cleansing phase, a benefit phase, a non-lathering structured aqueous phase and combinations thereof.

22. The multi-phase personal care composition of claim 20, wherein said pattern is selected from the group consisting of striped, geometric, marbled and combinations thereof.

23. The multi-phase personal care composition of claim 20, comprising at least about 0.1% by weight of the composition, of said particle,

24. The multi-phase personal care composition of claim 20, wherein said particle is selected from the group consisting of natural, synthetic, semi-synthetic, hybrid and combinations thereof.

25. The multi-phase personal care composition of claim 20, wherein said particle is selected from the group consisting of an exfoliant particle, a shiny particle, and combinations thereof.

26. The multi-phase personal care composition of claim 25, wherein said shiny particle is an interference pigment; wherein said interference pigment is a hydrophobically modified interference pigment; and wherein said composition comprises at least about 0.1% by weight of said composition, of said hydrophobically modified interference pigment.

27. The multi-phase personal care composition of claim 20, wherein said composition further comprises a bead.

28. The multi-phase personal care composition of claim 20, wherein said composition further comprises a skin lightening agent.

29. A multi-phase personal care composition comprising:
a) a first phase comprising a cleansing phase comprising from about 1% to about 50%, by weight of said cleansing phase, of a surfactant selected from the group consisting of anionic surfactant, nonionic surfactant, zwitterionic surfactant, cationic surfactant, soap, and mixtures thereof;
wherein said cleansing phase is non-Newtonian shear thinning, has a viscosity of equal to or greater than about 3,000 cps, and/or has a Yield Point of at least about 0.1 Pa;
b) a benefit phase comprising a hydrophobic composition comprising from about 20% to about 100%, by weight of said benefit phase of a hydrophobic material is selected from the group consisting of lipids, hydrocarbons, fats, oils, hydrophobic plant extracts, fatty acids, essential oils, silicone oils, and mixtures thereof;
wherein said benefit phase comprising a hydrophobic composition has a Vaughan Solubility Parameter of from about 5 to about 15 and further wherein a weight ratio between said cleansing phase and said benefit phase is from about 1:9 to about 99:1 and said cleansing phase and benefit phase are in physical contact in the same package and remain stable in ambient conditions for at least about 180 days; and wherein said cleansing phase and benefit phase form a striped pattern having a stripe size at least about 0.1 mm in width and at least about 1 mm in length; and wherein at least one phase comprises a particle; wherein said particle is a hydrophobically modified interference pigment.

30. The multi-phased personal care composition of claim 29, wherein said benefit phase is substantially free of surfactant.

31. The multi-phased personal care composition of claim 29, comprising at least about 0.1 weight percent by weight of the composition, of said hydrophobically modified interference pigment.

32. The striped phase personal cleansing composition of claim 29, wherein said hydrophobic material is selected from the group consisting of petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and combinations thereof.

33. The multi-phase personal care composition of claim 29, wherein said composition further comprises a bead.

34. The multi-phase personal care composition of claim 29, wherein said composition further comprises a skin lightening agent.

35. The multi-phase personal care composition of claim 1, wherein at least one phase comprises a colorant.

36. The multi-phase personal care composition of claim 35, wherein said composition is packaged in a transparent container.

37. The multi-phase personal cleansing composition of claim 1, wherein said composition additionally comprises skin care actives, wherein the skin care actives are selected from the group consisting of vitamins and derivatives thereof; sunscreens; preservatives; anti-acne medicaments; antioxidants; skin soothing and healing; chelators and sequestrants; essential oils, skin sensates, and mixtures thereof.

38. A multi-phase personal care composition comprising:
a) a first phase comprising a cleansing phase comprising from about 1% to about 50%, by weight of the cleansing phase, of a surfactant selected from the group consisting of anionic surfactant, non-ionic surfactant, zwitterionic surfactant, cationic surfactant, soap and mixtures thereof;
wherein said cleansing phase is non-Newtonian shear thinning, has a viscosity of equal to or greater than about 3,000 cps and/or a Yield Point of at least about 0.1 Pa; and
b) at least one additional phase comprising a separate non-lathering structured aqueous phase having a consistency value of at least 10 poise/(1/s) and a Yield Point of at least about 0.1 Pa; and
wherein at least one phase comprises a particle; wherein said particle is a hydrophobically modified interference pigment; wherein the ratio of the cleansing phase to the non-lathering structured aqueous phase is from about 10:1 to about 1:10;
wherein the cleansing phase and non-lathering structured aqueous phase are present as a pattern; wherein said pattern is a striped pattern; wherein the stripe size is at least about 0.1 mm in width and at least about 1 mm in length.

39. A method of delivering skin benefits to the skin or hair, said method comprising the steps of:
a) dispensing an effective amount of a composition of claim 1 onto an implement selected from the group consisting of a cleansing puff, washcloth, sponge and human hand;
b) topically applying said composition to the skin or hair using said implement; and
c) removing said composition from the skin or hair by rinsing the skin or hair with water.
